(19) United States
(12) Patent Application Publication
(10) Pub. No.: US 2014/0350181 A1
(43) Pub. Date: Nov. 27, 2014

(54) HYDRO-FLUOROCOMPOUNDS

(71) Applicants: Vito Tortelli, Milano (IT); Ivan Wlassics, Garessio (IT); Cristiano Monzani, Trezzo Sull'adda (IT); Bradley Lane Kent, US-Woolwich Township, NJ (US); Alessandro Veneroni, Novate Milanese (IT)

(72) Inventors: Vito Tortelli, Milano (IT); Ivan Wlassics, Garessio (IT); Cristiano Monzani, Trezzo Sull'adda (IT); Bradley Lane Kent, US-Woolwich Township, NJ (US); Alessandro Veneroni, Novate Milanese (IT)

(73) Assignee: SOLVAY SPECIALTY POLYMERS ITALY S.P.A., Bollate (IT)

(21) Appl. No.: 14/118,312

(22) PCT Filed: May 24, 2012
(86) PCT No.: PCT/EP12/59702

§ 371 Date: Jan. 31, 2014

Related U.S. Application Data
(60) Provisional application No. 61/490,147, filed on May 26, 2011.

(30) Foreign Application Priority Data
Aug. 3, 2011 (EP) 11176424.7

(51) Int. Cl. C07C 59/135; C08K 3/20; C07C 51/377

(52) U.S. Cl. CPC C07C 59/135; C07C 51/377; C08K 3/20
USPC 524/845; 562/586; 252/182.15

(57) ABSTRACT

The invention pertains to certain hydro-fluorocompounds of the following formula (I): R_{f}O—R_{H}—O—(CH2)ₘ-[CF(X)ₙ]—COOXₐ wherein: —Xₐ is H, a monovalent metal (preferably an alkaline metal) or an ammonium group of formula —N(R′ₙ)₄, wherein each of R′ₙ, equal to or different from each other, independently represents a hydrogen atom or a C₁₋₆ hydrocarbon group (preferably an alkyl group); —R_{f} is a C₁-C₆ (per)fluoroalkyl optionally comprising one or more catenary oxygen atoms, preferably R_{f} is a group of formula R_{f}—CH₂—, wherein R′_{f} is a C₁-C₅ perfluorinated group, possibly comprising one or more ethereal oxygens, preferably a C₁-C₃ perfluorinated group, possibly comprising one or more ethereal oxygens; —R_{H} is a fluorine-free hydrocarbon group optionally comprising one or more catenary oxygen atoms; —X is F or CF₃, preferably X is F; -m is 0 or 1; -n is 1 to 3, to a process for the manufacture of said hydro-fluorocompounds, to a method of making fluoropolymers in the presence of said hydro-fluorocompounds, and to fluoropolymer dispersions comprising said hydro-fluorocompound.

### CROSS-REFERENCE TO RELATED APPLICATIONS

This application claims priority to U.S. Provisional Application No. 61/490,147 filed May 26, 2011 and European application No. 11176424.7 filed Aug. 3, 2011, the whole content of these applications being incorporated herein by reference for all purposes.

### TECHNICAL FIELD

The present invention pertains to certain fluorosurfactants, to a method for manufacturing the same, to a method of making fluoropolymer dispersions using said fluorosurfactants, and to fluoropolymer dispersions therefrom.

### BACKGROUND ART

Fluoropolymers, i.e. polymers having a fluorinated backbone, have been long known and have been used in a variety of applications because of several desirable properties such as heat resistance, chemical resistance, weatherability, UV-stability etc.

A frequently used method for producing fluoropolymers involves aqueous emulsion polymerization of one or more fluorinated monomers generally involving the use of fluorinated surfactants. Frequently used fluorinated surfactants include perfluorooctanoic acids and salts thereof, in particular ammonium perfluorooctanoic acid.

Recently, perfluoroalkanoic acids having 8 or more carbon atoms have raised environmental concerns. For instance, perfluoroalkanoic acids have been found to show bioaccumulation. Accordingly, efforts are now devoted to phasing out from such compounds and methods have been developed to manufacture fluoropolymer products using alternative surfactants having a more favourable toxicological profile.

Several approaches have been recently pursued to this aim, typically involving fluorosurfactants comprising a perfluoroalkyl chain interrupted by one or more catenary oxygen atoms, said chain having an ionic carboxylate group at one of its ends.

Examples of these compounds which are endowed with improved bioaccumulation profile over perfluoro alkanoic acids having 8 or more carbon atoms can be found notably in US 2007276103 (3M INNOVATIVE PROPERTIES CO) Nov. 29, 2007, US 2007015864 (3M INNOVATIVE PROPERTIES CO) Jan. 18, 2007, US 2007015865 (3M INNOVATIVE PROPERTIES CO) Jan. 18, 2007, US 2007015866 (3M INNOVATIVE PROPERTIES CO) Jan. 18, 2007.

It would thus be desirable to find alternative fluorinated surfactants that can be used in the emulsion polymerization of fluorinated monomers which desirably show lower bioaccumulation/bio-persistence than perfluoro alkanoic acids having 8 or more carbon atoms.

It would further be desirable that the surfactant properties of said alternative fluorinated surfactants be such that polymerization can be carried out in a convenient and cost effective way, using equipment commonly used in the aqueous emulsion polymerization of fluorinated monomers with traditional surfactants.

### DISCLOSURE OF INVENTION

It has been found that hydro-fluorocompounds of the following formula (I) as below detailed, comprising in their structure:
a fluorinated group in remote position with respect to the anionic group;
a segregated fluorine-free hydrogenated moiety comprised between two ethereal oxygen atoms, linked to said fluorinated group and to a carboxylate-containing group through said oxygen atoms; and
a carboxylate group having a fluorinated group in alpha position; are effective in the aqueous emulsion polymerization of fluoromonomers, in particular of vinylidene fluoride, even when used without the addition of other surfactants such as perfluoroalkanoic acids and salts thereof.

Moreover, the Applicant has surprisingly found that above mentioned hydro-fluorocompounds (I) have significantly improved bio-persistence behaviour over perfluoroalkanoic acids derivatives, so that their toxicological profile is much improved.

Thus, in one aspect, the invention relates to hydro-fluorocompounds of formula (I):

R_{f}O—R_{H}—O—(CH₂)ₘ—[CF(X)]ₙ—COOXₐ

wherein:
Xₐ is H, a monovalent metal (preferably an alkaline metal) or an ammonium group of formula —N(R′ₙ)₄, wherein each of R′ₙ, equal to or different from each other, independently represents a hydrogen atom or a C₁₋₆ hydrocarbon group (preferably an alkyl group);
R_{f} is a C₁-C₆ (per)fluoroalkyl optionally comprising one or more catenary oxygen atoms, preferably R_{f} is a group of formula R′_{f}—CH₂—, wherein R′_{f} is a C₁-C₅ perfluorinated group, possibly comprising one or more ethereal oxygens, preferably a C₁-C₃ perfluorinated group, possibly comprising one or more ethereal oxygens;
R_{H} is a fluorine-free hydrocarbon group optionally comprising one or more catenary oxygen atoms;
X is F or CF₃, preferably X is F;
m is 0 or 1;
n is 1 to 3.

A process for manufacturing said hydro-fluorocompounds of the formula (I) is another object of the present invention.

Further, in one other aspect, the invention relates to a method for making a fluoropolymer comprising an aqueous emulsion polymerization of one or more fluorinated monomers wherein said aqueous emulsion polymerization is carried out in an aqueous medium comprising at least one hydro-fluorocompounds of the formula (I), as above detailed.

The hydro-fluorocompounds of formula (I) can be manufactured generally via multi-step synthetic pathways, applying to certain precursors known organic chemistry reactions, to provide said compounds.

The hydro-fluorocompounds of the invention and suitable for being used in the method for making a fluoropolymer, as above detailed, preferably comply with formula (II):

R_{f}O—(CH₂)ₚ—O—(CH₂)ₘ—[CF(X)]ₙ—COOXₐ

wherein R_{f}, X, Xₐ, m, n have the same meaning as above detailed, and p is an integer of 1 to 12, preferably of 2 to 10, including 2, 4, 6, 8.

Still more preferably, the hydro-fluorocompounds comply with formula (III):

R′_{f}—CH₂O—(CH₂)ₚ—O—(CH₂)ₘ—[CF(X)]ₙ—COOXₐ

wherein X, Xₐ, m, n, p have the same meaning as above detailed, and R′_{f} is a C₁-C₅ perfluorinated group, possibly comprising one or more ethereal oxygens, preferably a C₁-C₃ perfluorinated group, possibly comprising one or more ethereal oxygens.

According to a first embodiment, the hydro-fluorocompounds preferably comply with formula (IV-A):

R_{f}O—(CH₂)ₚ—O—CH₂—CF(X)—COOXₐ

wherein R_{f}, Xₐ, p have the same meaning as above detailed.

Hydro-fluorocompounds according to this embodiment include notably CF₃CH₂—O—(CH₂)₂—O—CH₂—CF₂—COOXₐ, CF₃CH₂—O—(CH₂)₄—O—CH₂—CF₂—COOXₐ, with Xₐ having the meaning as above detailed.

Compounds of formula (IV-A), wherein p=2, can be notably manufactured by reacting an alcohol of formula R_{f}OH, with R_{f} being as above defined, with ethylene carbonate, for obtaining hydroxyl derivative of formula R_{f}O—(CH₂)₂—OH, and subsequent reaction thereof with a fluorinated oxetane derivative of formula:

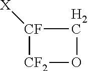

with X═F, CF₃, typically with 2,2,3,3-tetrafluorooxetane, to yield, after hydrolysis and neutralization, the carboxylic derivative R_{f}O—(CH₂)₂—O—CH₂—CFX—COOH, which might be salified, as required.

As an alternative, compounds (IV-A) can be manufactured by reaction of a ω-halo-hydroxy-derivative of formula Hal-(CH₂)ₚ—OH, wherein Hal is a halogen, typically Cl, and p has the meaning as above detailed (p is an integer of 1 to 12, preferably of 2 to 10, including 2, 4, 6, 8) with a fluorinated oxetane derivative of formula:

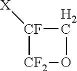

with X═F, CF₃, typically with 2,2,3,3-tetrafluorooxetane, to yield a derivative of formula Hal-(CH₂)ₚ—OCH₂—CF(X)—C(O)—O—(CH₂)ₚ-Hal. Subsequent reaction thereof with the alcoholate form of a fluorinated alcohol of formula R_{f}—OH, with R_{f} as above detailed, advantageously provides, after hydrolysis and neutralization, for the carboxylic derivative of formula R_{f}—O—(CH₂)ₚ—OCH₂—CF(X)—COOH which can be salified, as required.

According to a second embodiment, the hydro-fluorocompounds preferably comply with formula (IV-B):

R_{f}O—(CH₂)ₚ—O—CF₂—CF₂—COOXₐ

wherein R_{f}, Xₐ, p have the same meaning as above detailed.

Hydro-fluorocompounds according to this embodiment include notably CF₃CH₂—O—(CH₂)₂—O—CF₂—CF₂—COOXₐ, CF₃CH₂—O—(CH₂)₄—O—CF₂—CF₂—COOXₐ, CF₃CH₂—O—(CH₂)₆—O—CF₂—CF₂—COOXₐ, CF₃CH₂—O—(CH₂)₈—O—CF₂—CF₂—COOXₐ, with Xₐ having the meaning as above detailed.

Hydro-fluorocompounds of formula (IV-B) can be obtained by reaction of a fluoroalcohol of formula R_{f}OH with a ω-halo-hydroxy-derivative of formula Hal-(CH₂)ₚ-OH, wherein Hal is a halogen, typically Cl, and p has the meaning as above detailed (p is an integer of 1 to 12, preferably of 2 to 10, including 2, 4, 6, 8), to advantageously yield corresponding adduct of formula R_{f}O—(CH₂)ₚ—OH. This adduct is then advantageously reacted with a mixture of tetrafluoroethylene and an alkylcarbonate to yield, after hydrolysis, corresponding carboxylic derivative of formula R_{f}O—(CH₂)ₚ—O—CF₂CF₂—COOH, which can be further salified if needed.

In the method of making a fluoropolymer, one or more hydro-fluorocompounds of formula (I) are used in the aqueous emulsion polymerization of one or more fluorinated monomers, in particular gaseous fluorinated monomers.

By gaseous fluorinated monomers is meant monomers that are present as a gas under the polymerization conditions. In a particular embodiment, the polymerization of the fluorinated monomers is started in the presence of the hydro-fluorocompound of formula (I), i.e. the polymerization is initiated in the presence of the same. The amount of hydro-fluorocompound of formula (I) used may vary depending on desired properties such as amount of solids, particle size etc. . . . Generally the amount of hydro-fluorocompound of formula (I) will be between 0.001% by weight based on the weight of water in the polymerization and 5% by weight. A practical range is between 0.05% by weight and 1% by weight.

The skilled in the art will generally select the most appropriate concentration of hydro-fluorocompound of formula (I) in order to tune the average particle size of the fluoropolymer particles which are intended to be manufactured.

While the polymerization is generally initiated in the presence of the hydro-fluorocompound of formula (I), it is not excluded to add further hydro-fluorocompound of formula (I) during the polymerization, although such will generally not be necessary.

Nevertheless, it may be desirable to add certain monomer to the polymerization in the form of an aqueous emulsion. For example, fluorinated monomers that are liquid under the polymerization conditions may be advantageously added in the form of an aqueous emulsion. Such emulsion of such co-monomers is preferably prepared using hydro-fluorocompound of formula (I) as an emulsifier.

The aqueous emulsion polymerization may be carried out at a temperature between 10° C. to 150° C., preferably 20° C. to 130° C. and the pressure is typically between 2 and 50 bar, in particular 5 to 35 bar.

The reaction temperature may be varied during the polymerization e.g. for influencing the molecular weight distribution, i.e., to obtain a broad molecular weight distribution or to obtain a bimodal or multimodal molecular weight distribution.

The pH of the polymerization media may be in the range of pH 2-11, preferably 3-10, most preferably 4-10.

The aqueous emulsion polymerization is typically initiated by an initiator including any of the initiators known for initiating a free radical polymerization of fluorinated monomers. Suitable initiators include peroxides and azo compounds and redox based initiators. Specific examples of peroxide initiators include, hydrogen peroxide, sodium or barium peroxide, diacylperoxides such as diacetylperoxide, disuccinyl peroxide, dipropionylperoxide, dibutyrylperoxide, dibenzoylperoxide, di-ter-butyl-peroxide, benzoylacetylperoxide, diglutaric acid peroxide and dilaurylperoxide, and further per-acids and salts thereof such as e.g. ammonium, sodium or potassium salts. Examples of per-acids include peracetic acid. Esters of the peracid can be used as well and examples thereof include tert.-butylperoxyacetate and tert.-butylperoxypivalate. Examples of inorganic initiators include for example ammonium-alkali- or earth alkali salts of persulfates, permanganic or manganic acid or manganic acids. A persulfate initiator, e.g. ammonium persulfate (APS), can be used on its own or may be used in combination with a reducing agent. Suitable reducing agents include bisulfites such as for example ammonium bisulfite or sodium metabisulfite, thiosulfates such as for example ammonium, potassium or sodium thiosulfate, hydrazines, azodicarboxylates and azodicarboxyldiamide (ADA). Further reducing agents that may be used include hydroxymethane sodium sulfinate (Rongalite) or fluoroalkyl sulfinates such as those disclosed in U.S. Pat. No. 5,285,002. The reducing agent typically reduces the half-life time of the persulfate initiator. Additionally, a metal salt catalyst such as for example copper, iron or silver salts may be added.

The amount of initiator may be between 0.01% by weight (based on the fluoropolymer to be produced) and 1% by weight. Still, the amount of initiator is preferably between 0.05 and 0.5% by weight and more preferably between 0.05 and 0.3% by weight.

The aqueous emulsion polymerization can be carried out in the presence of other materials, such as notably buffers and, if desired, complex-formers or chain-transfer agents.

Examples of chain transfer agents that can be used include dimethyl ether, methyl t-butyl ether, alkanes having 1 to 5 carbon atoms such as ethane, propane and n-pentane, halogenated hydrocarbons such as CCl₄, CHCl₃ and CH₂Cl₂ and hydrofluorocarbon compounds such as CH₂F—CF₃ (R134a). Additionally esters like ethylacetate, malonic esters can be effective as chain transfer agent in the method of the invention.

Examples of fluorinated monomers that may be polymerized using the hydro-fluorocompound according to formula (I) as an emulsifier in the process of the invention include partially or fully fluorinated monomers including fluorinated olefins such as tetrafluoroethylene (TFE), chlorotrifluoroethylene (CTFE), hexafluoropropylene (HFP), vinyl fluoride (VF), vinylidene fluoride (VDF), partially or fully fluorinated allyl ethers and partially or fully fluorinated alkyl or alkoxy-vinyl ethers. The polymerization may further involve non-fluorinated monomers such as ethylene and propylene.

The method of the present invention may be used to produce a variety of fluoropolymers including perfluoropolymers, which have a fully fluorinated backbone, as well as partially fluorinated fluoropolymers. Also the method of the invention may result in melt-processable fluoropolymers as well as those that are not melt-processable such as for example polytetrafluoroethylene and so-called modified polytetrafluoroethylene. The method of the invention can further yield fluoropolymers that can be cured to make fluoroelastomers as well as fluorothermoplasts. Fluorothermoplasts are generally fluoropolymers that have a distinct and well noticeable melting point, typically in the range of 60 to 320° C. or between 100 and 320° C. They thus have a substantial crystalline phase. Fluoropolymers that are used for making fluoroelastomers typically are amorphous and/or have a negligible amount of crystallinity such that no or hardly any melting point is discernable for these fluoropolymers.

The Applicant has found that hydro-fluorocompound according to formula (I) are particularly effective for manufacturing thermoplastic vinylidene fluoride polymers by polymerizing vinylidene fluoride (VDF) optionally in combination with one or more fluorinated monomers different from VDF.

Further, the method of the invention can be carried out in the presence of fluorinated fluids, typically enabling formation of nanosized droplets (average size of less than 50 nm, preferably of less than 30 nm) stabilized in aqueous dispersion by the presence of the hydro-fluorocompound of formula (I).

Should the method of the invention be carried out in the presence of a fluorinated fluid, as above detailed, it may be preferable to first homogenously mix hydro-fluorocompound as above detailed and said fluid in aqueous phase, possibly in an aqueous medium, and then feeding an aqueous mixture of hydro-fluorocompound as above detailed and said fluid in the polymerization medium. This technique is particularly advantageous as this pre-mix can advantageously enable manufacture of an emulsion of said fluid in an aqueous phase comprising the hydro-fluorocompound as above detailed, wherein this emulsion comprises advantageously dispersed droplets of said fluid having an average size of preferably less than 50 nm, more preferably of less than 40 nm, even more preferably of less than 30 nm.

Fluids which can be used according to this embodiment are preferably (per)fluoropolyethers comprising recurring units (R1), said recurring units comprising at least one ether linkage in the main chain and at least one fluorine atom (fluoropolyoxyalkylene chain). Preferably the recurring units R1 of the (per)fluoropolyether are selected from the group consisting of:

(I) —CFX—O—, wherein X is F or CF₃; and

(II) —CF₂—CFX—O—, wherein X is F or CF₃; and

(III) —CF₂—CF₂—CF₂—O—; and

(IV) —CF₂—CF₂—CF₂—CF₂—O—; and

(V) —(CF₂)ⱼ—CFZ—O— wherein j is an integer chosen from 0 and 1 and Z is a fluoropolyoxyalkylene chain comprising from 1 to 10 recurring units chosen among the classes (I) to (IV) here above; and mixtures thereof.

Should the (per)fluoropolyether comprise recurring units R1 of different types, advantageously said recurring units are randomly distributed along the fluoropolyoxyalkylene chain.

Preferably the (per)fluoropolyether is a compound complying with formula (I-p) here below:

T₁-(CFX)ₚ—O—R_{f}—(CFX)_{p′}-T₂ (I-p)

wherein:
each of X is independently F or CF₃;
p and p′, equal or different each other, are integers from 0 to 3;
R_{f} is a fluoropolyoxyalkylene chain comprising repeating units R°, said repeating units being chosen among the group consisting of:

(i) —CFXO—, wherein X is F or CF₃,

(ii) —CF₂CFXO—, wherein X is F or CF₃,

(iii) —CF₂CF₂CF₂O—,

(iv) —CF₂CF₂CF₂CF₂O—,

(v) —(CF₂)ⱼ—CFZ—O— wherein j is an integer chosen from 0 and 1 and Z is a group of general formula —OR_{f}′T₃, wherein R_{f}′ is a fluoropolyoxyalkene chain comprising a number of repeating units from 0 to 10, said recurring units being chosen among the followings: —CFXO—, —CF₂CFXO—, —CF₂CF₂CF₂O—, —CF₂CF₂CF₂CF₂O—, with each of each of X being independently F or CF₃; and T₃ is a C₁-C₃ perfluoroalkyl group, and mixtures thereof;
T₁ and T₂, the same or different each other, are H, halogen atoms, C₁-C₃ fluoroalkyl groups, optionally comprising one or more H or halogen atoms different from fluorine.

According to an embodiment of the method of the invention, the method comprises polymerizing in aqueous emulsion in the presence of a mixture of the hydro-fluorocompound of formula (I) and at least one further emulsifier different from the hydro-fluorocompound of formula (I).

The choice of said additional emulsifier is not particularly limited. Both fluorine-free and fluorinated emulsifiers can be used in combination with hydro-fluorocompound of formula (I).

More specifically, fluorinated emulsifier [surfactant (FS)] of formula:

R_{f§}(X⁻)ⱼ(M⁺)ⱼ

wherein R_{f§} is a C₃-C₃₀ (per)fluoroalkyl chain, (per)fluoro(poly)oxyalkylenic chain, X⁻ is —COO⁻, —PO₃⁻ or —SO₃⁻, M⁺ is selected from H⁺, NH₄⁺, an alkaline metal ion and j can be 1 or 2 can be used.

As non limitative example of surfactants (FS), mention may be made of ammonium and/or sodium perfluorocarboxylates, and/or (per)fluoropolyoxyalkylenes having one or more carboxylic end groups.

Other examples of fluorinated surfactants are (per)fluorooxyalkylenic surfactants described in US 2007015864 (3M INNOVATIVE PROPERTIES) Jan. 8, 2007 , US 2007015865 (3M INNOVATIVE PROPERTIES CO) Jan. 18, 2007, US 2007015866 (3M INNOVATIVE PROPERTIES CO) Jan. 18, 2007, US 2007025902 (3M INNOVATIVE PROPERTIES CO) Feb. 1, 2007.

More preferably, the fluorinated emulsifier [surfactant (FS)] is chosen from:
CF₃(CF₂)ₙ₁COOM′, in which n₁ is an integer ranging from 4 to 10, preferably from 5 to 7, and more preferably being equal to 6; M′ represents H, NH₄, Na, Li or K, preferably NH₄;
T(C₃F₆O)ₙ₀(CFXO)ₘ₀CF₂COOM″, in which T represents Cl or a perfluoroalkoxyde group of formula CₖF₂ₖ₊₁O with k is an integer from 1 to 3, one F atom being optionally substituted by a Cl atom; n₀ is an integer ranging from 1 to 6; m₀ is an integer ranging from 0 to 6; M″ represents H, NH₄, Na, Li or K; X represents F or CF₃;
F—(CF₂—CF₂)ₙ₂—CH₂—CH₂—RO₃M′″, in which R is P or S, preferably S, M′″ represents H, NH₄, Na, Li or K, preferably H; n₂ is an integer ranging from 2 to 5, preferably n₂=3;
A-R_{f}—B bifunctional fluorinated surfactants, in which A and B, equal to or different from each other, are —(O)ₚCFX—COOM*; M* represents H, NH₄, Na, Li or K, preferably M* represents NH₄; X═F or CF₃; p is an integer equal to 0 or 1; R_{f} is a linear or branched perfluoroalkyl chain, or a (per)fluoropolyether chain such that the number average molecular weight of A-R_{f}—B is in the range 300 to 3,000, preferably from 500 to 2,000;
R′_{f}—O—(CF₂)ᵣ—O-L-COOM′, wherein R′_{f} is a linear or branched perfluoroalkyl chain, optionally comprising catenary oxygen atoms, M′ is H, NH₄, Na, Li or K, preferably M′ represents NH₄; r is 1 to 3; L is a bivalent fluorinated bridging group, preferably —CF₂CF₂— or —CFX—, X═F or CF₃;
R″_{f}—(OCF₂)ᵤ—O—(CF₂)ᵥ—COOM″, wherein R″_{f} is a linear or branched perfluoroalkyl chain, optionally comprising catenary oxygen atoms, M″ is H, NH₄, Na, Li or K, preferably M″ represents NH₄; u and v are integers from 1 to 3;
R′″_{f}—(O)ₜ—CHQ-L-COOM′″, wherein R′″_{f} is a linear or branched perfluoroalkyl chain, optionally comprising catenary oxygen atoms, Q=F or CF₃, t is 0 or 1, M” is H, NH₄, Na, Li or K, preferably M′″ is NH₄; L is a bivalent fluorinated bridging group, preferably —CF₂CF₂— or —CFX—, X═F or CF₃;
and mixtures thereof.

Particular good results have been obtained with mixtures of hydro-fluorocompound of formula (I) with A-R_{f}—B bifunctional fluorinated surfactants; said bifunctional surfactant A-R_{f}—B preferably complies with formula M_{z}OOC—CFX_{z}—O—R_{fz}—CFX_{z}—COOM_{z}, wherein M_{z} is H, NH₄, Na, Li or K, preferably M_{z} is NH₄; X_{z}═F, —CF₃; R_{fz} is a (per)fluoropolyether chain comprising recurring units complying with one or more of formulae: —(C₃F₆O)—; —(CF₂CF₂O)—; —(CFL_{O}O)—, wherein L₀=F, —CF₃; —(CF₂(CF₂)_{z′}CF₂O)—, wherein z′ is 1 or 2; —(CH₂CF₂CF₂O)—.

R_{fz} preferably has one of the following structures:

1) —(CF₂O)ₐ—(CF₂CF₂O)_{b}—

wherein a and b≧0; should a and b be simultaneously >0, b/a ratio is generally comprised between 0.01 and 10, extremes included;

2) —(CF₂—(CF₂)_{z′}—CF₂O)_{b′}—, with b′>0 and z′ being 1 or 2;

3) —(C₃F₆O)ᵣ—(C₂F₄O)_{b}—(CFL₀O)ₜ—, wherein r, b and t≧0, L₀=F, —CF₃; should r, b and t be simultaneously >0, r/b ratio is typically comprised in the range 0.5-2.0 and (r+b)/t in the range 10-30;

4) —(OC₃F₆)ᵣ—(OCFL₀)ₜ-OCF₂—R*_{f}—CF₂O—(C₃F₆O)ᵣ—(CFL₀O)ₜ—, wherein R*_{f} is a fluoroalkylene group from 1 to 4 carbon atoms; L₀=F, —CF₃; r, t being ≧0.

Most preferred A-R_{f}—B bifunctional fluorinated surfactant complies with formula M_{z}OOC—CFX_{z}—O—(CF₂O)ₐ—(CF₂CF₂O)_{b}—CFX_{z}—COOM_{z}, wherein M_{z} is H, NH₄, Na, Li or K, preferably M_{z} is NH₄; X_{z}═F, —CF₃; and a, b, both >0, are selected so that b/a is comprised between 0.3 and 10 and the molecular weight of the surfactant is comprised between 500 and 2000.

According to this embodiment, said A-R_{f}—B bifunctional fluorinated surfactant is preferably selected among compounds having a number average molecular weight of at least 1000 and a solubility in water of less than 1% by weight at 25° C. This selection generally provides for appropriate nucleating effect, enabling fine tuning of the particle size to be achieved. To this aim, said A-R_{f}—B bifunctional fluorinated surfactant is present in the aqueous medium of the polymerization process of the invention in an amount of 0.001 to 0.3 g/l. This embodiment is particularly advantageous for the manufacture of VDF polymers, as above detailed, of given particle sizes, e.g. suitable for coatings formulations.

Should the process of the invention be carried out in the presence of mixture of compound and further fluorinated emulsifier, as above detailed, it may be preferable to first homogenously mix hydro-fluorocompound according to formula (I) compound and further emulsifier in aqueous phase, and then feeding an aqueous mixture of compound (I) and said emulsifier in the polymerization medium. This technique is particularly advantageous when the further fluorinated emulsifier is poorly soluble in water. Thus, this pre-mix can advantageously enable manufacture of an emulsion of said additional fluorinated emulsifier in an aqueous phase comprising the hydro-fluorocompound according to formula (I) compound, wherein this emulsion comprises advantageously dispersed droplets of said fluorinated emulsifier having an average size of preferably less than 50 nm, preferably of less than 40 nm, more preferably of less than 30 nm.

Further, in addition, the aqueous emulsion polymerization of this embodiment can be carried out in the presence of fluorinated fluids, as above referred, typically enabling formation of nanosized droplets (average size of less than 50 nm, preferably of less than 30 nm) stabilized in aqueous dispersion by the presence of the mixture of the hydro-fluorocompound according to formula (I) and at least one further emulsifier different from fluorocompound of formula (I).

Fluorinated fluids which can be used in combination with said mixture of compound (I) and emulsifier are those above referred, suitable for being used in combination with the hydro-fluorocompound according to formula (I).

The method of the invention typically results in a aqueous dispersion of the fluoropolymer comprising the hydro-fluorocompound according to formula (I), which is another object of the present invention. Generally the amount of fluoropolymer in the dispersion directly resulting from the polymerization will vary between 3% by weight and about 40% by weight depending on the polymerization conditions. A typical range is between 5 and 35% by weight, preferably between 10 and 30% by weight.

The particle size (volume average diameter) of the fluoropolymer is typically between 40 nm and 400 nm with a typical particle size between 60 nm and about 350 nm being preferred. The total amount of hydro-fluorocompound according to formula (I) in the resulting dispersion is typically between 0.001 and 5% by weight based on the amount of fluoropolymer solids in the dispersion. A typical amount may be from 0.01 to 2% by weight or from 0.02 to 1% by weight.

The fluoropolymer may be isolated from the dispersion by coagulation if a polymer in solid form is desired. Also, depending on the requirements of the application in which the fluoropolymer is to be used, the fluoropolymer may be post-fluorinated so as to convert any thermally unstable end groups into stable CF₃— end groups.

For coating applications, an aqueous dispersion of the fluoropolymer is desired and hence the fluoropolymer will not need to be separated or coagulated from the dispersion. To obtain a fluoropolymer dispersion suitable for use in coating applications such as for example in the impregnation of fabrics or in the coating of metal substrates to make for example cookware, it will generally be desired to add further stabilizing surfactants and/or to further increase the fluoropolymer solids. For example, non-ionic stabilizing surfactants may be added to the fluoropolymer dispersion. Typically these will be added thereto in an amount of 1 to 12% by weight based on fluoropolymer solids. Examples of non-ionic surfactants that may be added include R¹—O—[CH₂CH₂O]ₙ—[R²O]ₘ—R³ (NS) wherein R¹ represents an aromatic or aliphatic hydrocarbon group having from 6 to 18 carbon atoms, R² represents an alkylene having 3 carbon atoms, R³ represents hydrogen or a O₁₋₃ alkyl group, n has a value of 0 to 40, m has a value of 0 to 40 and the sum of n+m being at least 2. It will be understood that in the above formula (NS), the units indexed by n and m may appear as blocks or they may be present in an alternating or random configuration. Examples of non-ionic surfactants according to formula (NS) above include alkylphenol oxy ethylates such as ethoxylated p-isooctylphenol commercially available under the brand name TRITON™ such as for example TRITON™ X 100 wherein the number of ethoxy units is about 10 or TRITON™ X 114 wherein the number of ethoxy units is about 7 to 8. Still further examples include those in which R¹ in the above formula (NS) represents an alkyl group of 4 to 20 carbon atoms, m is 0 and R³ is hydrogen. An example thereof includes isotridecanol ethoxylated with about 8 ethoxy groups and which is commercially available as GENAPOL® X080 from Clariant GmbH. Non-ionic surfactants according to formula (NS) in which the hydrophilic part comprises a block-copolymer of ethoxy groups and propoxy groups may be used as well. Such non-ionic surfactants are commercially available from Clariant GmbH under the trade designation GENAPOL® PF 40 and GENAPOL® PF 80.

The amount of fluoropolymer solids in the dispersion may be upconcentrated as needed or desired to an amount between 30 and 70% by weight. Any of the known upconcentration techniques may be used including ultrafiltration and thermal upconcentration.

Still an object of the invention are fluoropolymer dispersions comprising at least one hydro-fluorocompound according to formula (I), as above described.

Said fluoropolymer dispersions are typically obtained by the process of the invention.

Concentration of hydro-fluorocompound according to formula (I) in the fluoropolymer dispersions of the invention can be reduced, if necessary, following traditional techniques. Mention can be made of ultrafiltration combined with percolate recycle, as described in U.S. Pat. No. 4,369,266 (HOECHST AG) Jan. 18, 1983 , treatment with ion exchange resins in the presence of a non-ionic surfactant (as described in EP 1155055 A (DYNEON GMBH) Nov. 21, 2001), of an anionic surfactant (as exemplified in EP 1676868 A (SOLVAY SOLEXIS SPA) Jul. 5, 2006) or of a polyelectrolyte (as taught in EP 1676867 A (SOLVAY SOLEXIS SPA) Jul. 5, 2006).

The invention thus also pertains to a process for recovering hydro-fluorocompound according to formula (I) from fluoropolymer dispersions comprising the same. The process preferably comprises contacting the fluoropolymer dispersion with a solid adsorbing material, typically an ion exchange resin, preferably an anion exchange resin: the hydro-fluorocompound according to formula (I) is advantageously adsorbed (at least partially) onto the solid adsorbing material. The hydro-fluorocompound according to formula (I) can be efficiently recovered from solid adsorbing material by standard technique, including elution, thermal desorption and the like. In case of elution, in particular from anion exchange resin, hydro-fluorocompound according to formula (I) can be recovered by elution with an acidic solution. Typically, an aqueous medium comprising an acid and a water-miscible organic solvent can be used to this aim. Mixtures of inorganic acid and alcohol in water are particularly effective. The hydro-fluorocompound according to formula (I) can be notably recovered from such liquid phases by standard methods, including, notably crystallization, distillation (e.g. under the form of ester) and the like.

Also, hydro-fluorocompound according to formula (I) as above detailed and processes for its manufacture are other objects of the present invention.

The invention will be now explained in more detail with reference to the following examples, whose purpose is merely illustrative and not intended to limit the scope of the invention.

### PREPARATIVE EXAMPLE 1

Synthesis of CF₃—CH₂—O—(CH₂)₂—O—CH₂CF₂—COOH (P3)

Step 1.A—Synthesis of CF₃—CH₂—O—(CH₂)₂—OH (compound P1)

The compound has been synthesized according to the scheme herein below:

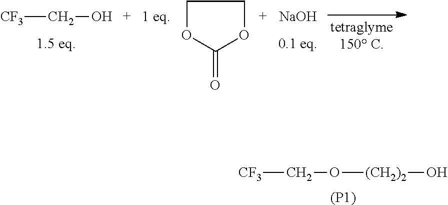

A solution comprising 47 g of ethylene carbonate and 80 g of 1,1,1-trifluoroethanol (corresponding to 1.5 molar excess over ethylene carbonate) were reacted in the presence of 0.1 molar equivalents of NaOH in 200 ml of tetraglyme at 150° C. during 4 hours. Target product was obtained with a selectivity of 100% moles and a yield of 86% moles. Product was further purified by distillation to obtain CF₃—CH₂—O—(CH₂)₂—OH (P1), in 76% moles yield.

Step 1.B—Synthesis of CF₃—CH₂—O—(CH₂)₂—O—CH₂CF₂—COOH (compound P3)

The compound has been synthesized according to the scheme herein below:

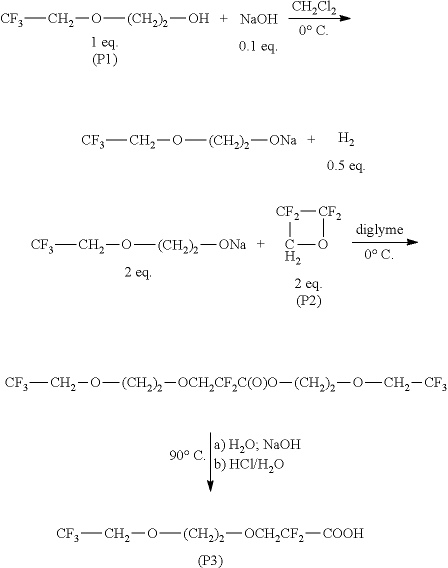

One molar equivalent of compound P1 was added drop-wise to a suspension of 0.99 eq. of NaH, suspended in a volume of CH₂Cl₂ to achieve a concentration equal to 1.7 M. Once evolution of hydrogen ended, salt of compound P1 was isolated by evaporating CH₂Cl₂ at 40° C. under reduced pressure (40 mmHg) for 1 hour. This salt was solubilised in anhydrous diglyme so as to obtain a solution having a concentration of 1.5 M; this solution was cooled at 0° C. and 2,2,3,3-tetrafluorooxetane (compound P2) was slowly added. The reaction mixture was let reverting to room temperature and maintained under stirring for 3 hours. Reaction mixture was then rinsed with water and an oily residue of ester CF₃—CH₂—O—(CH₂)₂—O—CH₂CF₂—C(O)O—(CH₂)₂—O—CH₂—CF₃ was isolated in 62% moles yield.

Said ester was directly hydrolyzed at 90° C. with aqueous NaOH (2 molar equivalents) for 60 minutes; the reaction mixture having basic pH (pH about 14) was submitted to evaporation at 60° C. under reduced pressure (30 mm Hg) in order to eliminate free alcohol CF₃—CH₂—O—(CH₂)₂—OH. Then, the residue was acidified in water comprising 5 molar equivalents of HCl at 80° C. for 1 hour. The mixture was then extracted 5 times with an equivalent volume of diethylether. The combined organic phases were evaporated obtaining as residue compound P3 in 47% moles yield with respect to P2.

### PREPARATIVE EXAMPLE 2

Synthesis of CF₃—CH₂—O—(CH₂)₄—O—CH₂CF₂—COOH (Q4)

Step 2.A—Synthesis of Cl—(CH₂)₄—O—CH₂CF₂—C(O)—O—(CH₂)₄—Cl (compound Q1)

The compound Q1 has been synthesized according to the scheme herein below:

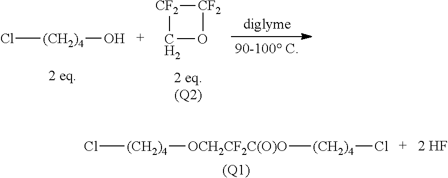

In a PTFE flask equipped with a refrigerant (maintained at −78° C.), a magnetic stirrer and a dropping funnel maintained at −5° C., a mixture of diglyme and Cl—(CH₂)₄—OH was introduced, so as to have a concentration of the chloroalcohol of 1 M. 2,2,3,3-tetrafluorooxetane (Q2) was then added drop-wise; at the end of the addition, the reaction mixture was heated to 90-100° C. After 32.5 hours, the conversion of Q2 was complete. Sodium carbonate was added in an amount of 1.5 eq. with respect to HF (as spectroscopically determined by NMR in crude reaction mixture), in the presence of MgSO₄. The oily residue was then filtered under vacuum; separated solids were further rinsed with diethylether and organic extracts were evaporated; residues were combined with oily filtrate to provide compound Q1 as diglyme solution. The yield in compound Q1 was found to be of about 60% moles, based on Q2.

Step 2.B—Synthesis of CF₃—CH₂—O—(CH₂)₄—O—CH₂CF₂—COOH (compound Q4)

The compound Q4 has been synthesized according to the scheme herein below:

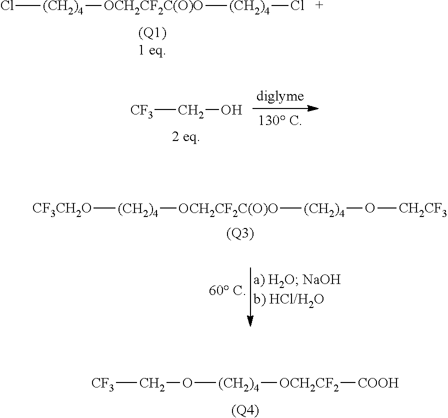

The diglyme solution containing compound Q1, as obtained from step 2.A herein above is added drop-wise to 2 molar equivalents of 1,1,1-trifluoroethanol in diglyme and the mixture was reacted at 130° C. for 10 hours, during which a white precipitate was formed. The precipitate was separated by centrifugation and rinsed with diethylether, obtaining, after evaporation of said diethylether, compound Q3 as solution in diglyme, with a 90% yield with respect to Q1.

Such solution was then acidified to a pH of 1 with sulphuric acid (96%) at 0° C. and extracted with three volumes of water for removing diglyme.

Oily residue of compound Q3 was then hydrolyzed with 2.2 eq. of Na₂CO₃, at a pH of 9.5-10 and at a temperature of 50-60° C. for 3.5 hours. The resulting homogeneous solution was then acidified with HCl to a pH of about 0.5. An oil precipitate was obtained which was further extracted with water to eliminate diglyme residues. Product Q4 was then isolated in a 45% moles yield with respect to Q2.

### PREPARATIVE EXAMPLE 3

Synthesis of CF₃—CH₂—O—(CH₂)₂—O—CF₂CF₂—COOH (R3)

Step 3.A—Synthesis of CF₃—CH₂—O—(CH₂)₂—OH (compound P1)

The compound was synthesized as detailed in Step 1.A of Preparative Example 1.

Step 3.B—Synthesis of CF₃-CH₂-0-(CH₂)₂-0-CF₂CF₂-COOH (compound

R3)

The compound was synthesized according to the scheme herein below:

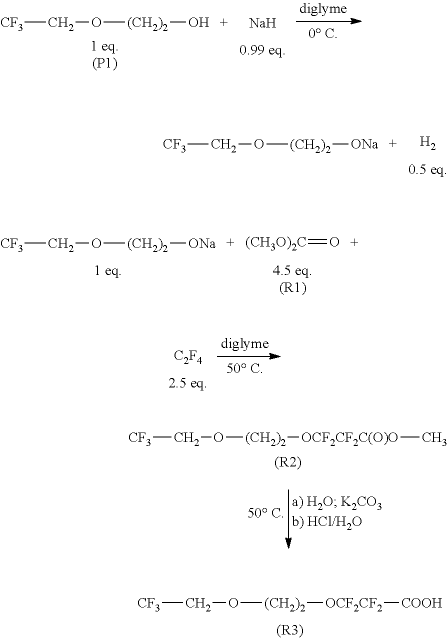

NaH (0.99 eq.) was suspended in diglyme in a volume such to give a concentration of 1.4 M. The mixture was cooled at 0° C. and 1 eq. of compound P1 was slowly added drop-wise avoiding temperature to rise beyond 5-6° C. Reaction was completed (no further H₂ evolution) after 2.5 hours, with a quantitative yield. The compound P1 so salified was transferred in an autoclave and cooled at −78° C. under vacuum; 4.5 molar equivalents of dimethylcarbonate (R1) and 2.5 equivalents of tetrafluoroethylene were then introduced in the cooled reactor, which was then let to warm to room temperature, and then heated at 50° C. for 15 hours. The conversion of sodium salt of compound (P1) was found to be about 98% moles. The crude reaction mixture was found to comprise compound (R2) as above detailed in admixture with CF₃—CH₂—O—(CH₂)₂ —O—CF₂CF₂—COONa, CF₃—CH₂—O—(CH₂)₂—O—CF₂CF₂—H, CH₃O—CF₂CF₂—COOCH₃ and CH₃O—CF₂CF₂—COONa. This crude mixture was cooled at 0° C. and acidified with H₂SO₄ 'til pH=1; the acidified mixture was then extracted with water to eliminate most of diglyme. Residual oil was then acidified with HCl at 90° C. to eliminate residual dimethylcarbonate, via decomposition. No acid hydrolysis of the compound (R2) was observed. The oil was then hydrolyzed with 2.1 molar eq. of K₂CO₃ in water at 20-25° C. for 2.5 hours, so as to limit decarboxylation. The organic phase, comprising notably all compounds with —CF₂H end-groups was removed. The aqueous phase was then acidified with aqueous HCl until a pH of 0.5, so as to effect precipitation of R3 (CF₃—CH₂—O—(CH₂)₂—O—CF₂CF₂—COOH); the aqueous phase was extracted twice with CH₂Cl₂ and the combined organic extracts, after evaporation of the solvent, were combined with the precipitated solid R3. Removal of CH₃O—CF₂CF₂—COOH, which preferentially remained in aqueous phase, was completed via fractional distillation under vacuum (0.6 mbar) at a temperature of 70-90° C. Compound R3 was isolated with 99.5% purity with a yield of 41% moles with respect to P1.

### PREPARATIVE EXAMPLE 4

Synthesis of CF₃—CH₂—O—(CH₂)₆—O—CF₂CF₂—COOH (S5)

Step 4.A—Synthesis of CF₃—CH₂—O—(CH₂)₆—OH (S2)

Compound S2 was synthesized according to the scheme herein below:

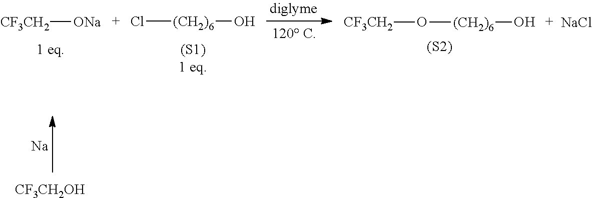

1,1,1-trifluoroethanol was salified with an excess of Na; the salified alcohol was then dissolved in diglyme so as to obtain a concentration of 2.5 M. The so obtained solution was heated at 120° C. and 1 eq. of compound S1 was added drop-wise. Conversion of S1 was completed after 5 hours reaction. Compound S2 was isolated solubilizing crude reaction mixture in a volume of water 2.2-fold larger than the crude volume. Then the mixture was acidified with aqueous HCl until a pH of about 1-2. Compound S2 precipitated with neat separation; aqueous phase was further extracted with CH₂Cl₂ and combined organic phases were rinsed with water, dried with MgSO4; after evaporation of the solvent, the residue was joined to the precipitated product to provide S2 with a 98.9% yield.

Step 4.B—Synthesis of CF₃—CH₂—O—(CH₂)₆—O—CF₂CF₂—COOH (S5)

Compound (S5) was synthesized according to the scheme herein below:

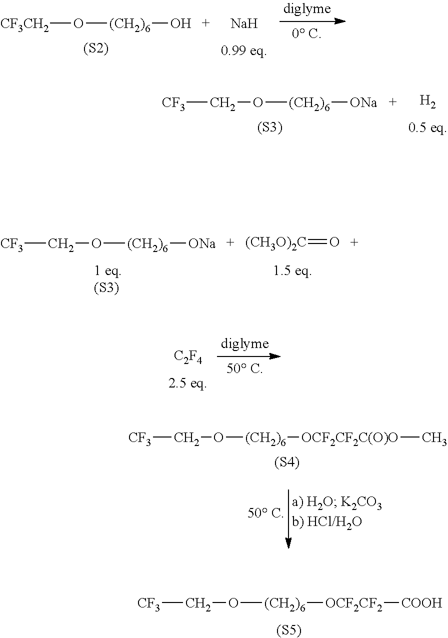

following procedure as detailed under section Step 3.B of preparative example 3, but using as starting material hydroxyl compound S2 instead of hydroxyl compound P1. Conversion of compound S2 was found to be 81.4% moles; overall yield in compound S5 with respect to S2 was found to be 63% moles, with a selectivity of 77.4% (because of decarboxylation phenomena leading to CF₃—CH₂—O—(CH₂)₆—O—CF₂CF₂—H). Nevertheless, purification provided for a final yield of pure S5 of about 45% (over S2).

### PREPARATIVE EXAMPLE 5

Synthesis of CF₃—CH₂—O—(CH₂)₈—O—CF₂CF₂—COOH (T5)

Step 5.A—Synthesis of CF₃—CH₂—O—(CH₂)₈—OH (T2)

Compound T2 was synthesized according to the scheme herein below:

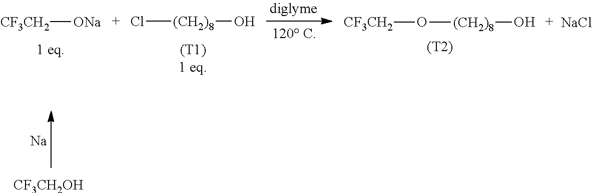

following similar procedure as detailed in Step 4.A of preparative Example 4 herein above, but using compound T1 instead of compound S1, and achieving complete conversion of the same after 6 hours. Compound T2 was obtained with a 90% yield and 100% selectivity.

Step 5.B—Synthesis of CF₃—CH₂—O—(CH₂)₈—O—CF₂CF₂—COON (T5)

Compound (T5) was synthesized according to the scheme herein below:

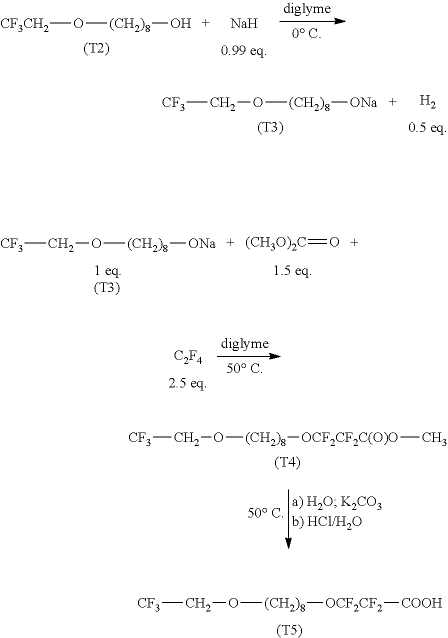

following same procedure as detailed for the manufacture of compound S5 under section Step 4.B of Preparative Example 4, but using compound T2 instead of compound S2. Conversion of compound T2 was found to be 80.3% moles; overall yield in compound T5 with respect to T2 was found to be 56.7% moles, with a selectivity of 70.6% (because of decarboxylation phenomena leading to CF₃—CH₂—O—(CH₂)₈—O—CF₂CF₂—H). Nevertheless, purification provided for a final yield of pure T5 of about 40% (over T2).

### POLYMERIZATION EXAMPLE 6

A 7.5-liter stainless steel horizontal reactor, equipped with a paddle agitator, was charged with a total of 5.375 kg of deionized water and an aqueous solution of the ammonium salt of CF₃CH₂O(CH₂)₂OCH₂CF₂COOH (product P3 obtained from Preparative Example 1) such that the concentration of the fluorosurfactant was 1.0 g/L in the aqueous phase of the reactor. In addition, 4 g of a hydrocarbon wax melting at 50 to 60° C. was added. The reactor was sealed and deaerated by heating with agitation to 100° C., then venting steam and air from the reactor for two minutes. The reactor was then heated to 122.5° C. Sufficient vinylidene fluoride monomer was introduced from a cylinder to bring the reactor pressure to 650 psig (44.8 bar). Then 24.4 mL of di-tert-butyl peroxide (DTBP) was pumped into the reactor to initiate the polymerization reaction. After an induction period of approximately 15 minutes, the reactor pressure decreased slightly, indicating initiation. Vinylidene fluoride then was continuously added as needed to maintain the reactor pressure at 650 psig (44.8 bar) while the reactor temperature was maintained at 122.5° C. by pumping water and ethylene glycol through the reactor jacket. After about 180 minutes, when a total of 1308 g of vinylidene fluoride had been fed to the reactor, the monomer feed was stopped. At that point, the reactor was cooled, the unreacted vinylidene fluoride was vented, and the latex was drained from the reactor. The resulting latex was analyzed by laser light scattering and found to have an average latex particle size of 244 nm.

### POLYMERIZATION EXAMPLE 7

The polymerization procedure in Example 6 was followed except for a decrease in CF₃CH₂O(CH₂)₂OCH₂CF₂COOH ammonium salt concentration to 0.7 g/L and the addition of sodium 1-octanesulfonate at a concentration of 1.2 g/L in the aqueous phase of the reactor. After about 315 minutes, when a total of 2298 g of vinylidene fluoride had been fed to the reactor, the monomer feed was stopped. In order to maximize yield, the system was allowed to continue reacting until the reactor pressure was decreased to about 150 psig (10.3 bar). At that point, the reactor was cooled, the unreacted vinylidene fluoride was vented, and the latex was drained from the reactor. The resulting latex was found to have an average particle size of 286 nm.

### POLYMERIZATION EXAMPLE 8

The polymerization procedure in Example 6 was followed except the ammonium salt of CF₃CH₂O(CH₂)₂OCF₂CF₂COOH (product R3 obtained from Preparative Example 3) was used with a concentration of 1.0 g/L in the aqueous phase of the reactor. After about 252 minutes, when a total of 1372 g of vinylidene fluoride had been fed to the reactor, the monomer feed was stopped. At that point, the reactor was cooled, the unreacted vinylidene fluoride was vented, and the latex was drained from the reactor. [Note: The latex was very unstable with about 92% of the polymer lost due to coagulation and suspension polymer formation. It was not possible to measure the particle size or other useful properties of the polymer.]

### POLYMERIZATION EXAMPLE 9

The polymerization procedure in Example 8 was followed except the CF₃CH₂O(CH₂)₂OCF₂CF₂COOH ammonium salt concentration was increased to 2.0 g/L and a bifunctional perfluoropolyether carboxylic acid of formula HOOC—CF₂O—(CF₂O)ₙ—(CF₂CF₂O)ₘ—CF₂COOH with an average molecular weight of 1800 was added at 10 mg/L in the aqueous phase of the reactor. After about 244 minutes, when a total of 1890 g of vinylidene fluoride had been fed to the reactor, the monomer feed was stopped. At that point, the reactor was cooled, the unreacted vinylidene fluoride was vented, and the latex was drained from the reactor.

1. Hydro-fluorocompound of formula (I):
R_{f}O—R_{H}—O—(CH₂)ₘ—[CF(X)]ₙ—COOXₐ
wherein:
Xₐ is H, a monovalent metal or an ammonium group of formula —N(R′ₙ)₄, wherein each of R′ₙ, equal to or different from each other, independently represents a hydrogen atom or a C₁₋₆ hydrocarbon group;
R_{f} is a C₁-C₆ (per)fluoroalkyl optionally comprising one or more catenary oxygen atoms or a group of formula R′_{f}—CH₂—, wherein R′_{f} is a C₁-C₅ perfluorinated group, optionally comprising one or more ethereal oxygens;
R_{H} is a fluorine-free hydrocarbon group optionally comprising one or more catenary oxygen atoms;
X is F or CF₃, preferably X is F;
m is 0 or 1; and
n is 1 to 3.

2. The hydro-fluorocompound of claim 1, said hydro-fluorocompound complying with formula (II):
R_{f}O—(CH₂)ₚ—O—(CH₂)ₘ—[CF(X)]ₙ—COOXₐ
wherein:
R_{f} is a C₁-C₆ (per)fluoroalkyl optionally comprising one or more catenary oxygen atoms or a group of formula R′_{f}—CH₂—, wherein R′_{f} is a C₁-C₅ perfluorinated group, optionally comprising one or more ethereal oxygens;
X is F or CF₃, preferably X is F;
Xₐ is H, a monovalent metal or an ammonium group of formula —N(R′ₙ)₄, wherein each of R′ₙ, equal to or different from each other, independently represents a hydrogen atom or a C₁₋₆ hydrocarbon group (preferably an alkyl group);
m is 0 or 1;
n is 1 to 3, and
p is an integer of 1 to 12.

3. The hydro-fluorocompound of claim 2, said hydro-fluorocompound complying with formula (III):
R′_{f}—CH₂O—(CH₂)ₚ—O—(CH₂)ₘ—[CF(X)]ₙ—COOXₐ
wherein:
X is F or CF₃, preferably X is F;
Xₐ is H, a monovalent metal or an ammonium group of formula —N(R′ₙ)₄, wherein each of R′ₙ, equal to or different from each other, independently represents a hydrogen atom or a C₁₋₆ hydrocarbon group (preferably an alkyl group);
m is 0 or 1;
n is 1 to 3, and
p is an integer of 1 to 12, and
R′_{f} is a C₁-C₅ perfluorinated group, optionally comprising one or more ethereal oxygens.

4. The hydro-fluorocompound of claim 2, said hydro-fluorocompound complying with formula (IV-A):
R_{f}O—(CH₂)ₚ—O—CH₂—CF(X)—COOXₐ
wherein:
R_{f} is a C₁-C₆ (per)fluoroalkyl optionally comprising one or more catenary oxygen atoms, or a group of formula R′_{f}—CH₂—, wherein R′_{f} is a C₁-C₅ perfluorinated group, optionally comprising one or more ethereal oxygens;
Xₐ is H, a monovalent metal or an ammonium group of formula —N(R′ₙ)₄, wherein each of R′ₙ, equal to or different from each other, independently represents a hydrogen atom or a C₁₋₆ hydrocarbon group (preferably an alkyl group); and
p is an integer of 1 to 12.

5. The hydro-fluorocompound of claim 4, selected from the group consisting of:
CF₃CH₂—O—(CH₂)₂—O—CH₂—CF₂—COOXₐ, and
CF₃CH₂—O—(CH₂)₄—O—CH₂—CF₂—COOXₐ,
wherein Xₐ is H, a monovalent metal or an ammonium group of formula —N(R′ₙ)₄, wherein each of R′ₙ, equal to or different from each other, independently represents a hydrogen atom or a C₁₋₆ hydrocarbon group.

6. The hydro-fluorocompound of claim 2, said hydro-fluorocompound complying with formula (IV-B):
R_{f}O—(CH₂)ₚ—O—CF₂—CF₂—COOXₐ
wherein:
R_{f} is a C₁-C₆ (per)fluoroalkyl optionally comprising one or more catenary oxygen atoms, or a group of formula R′_{f}—CH₂—, wherein R′_{f} is a C₁-C₅ perfluorinated group, optionally comprising one or more ethereal oxygens;
Xₐ is H, a monovalent metal (preferably an alkaline metal) or an ammonium group of formula N(R′ₙ)₄, wherein each of R′ₙ, equal to or different from each other, independently represents a hydrogen atom or a C₁₋₆ hydrocarbon group; and
p is an integer of 1 to 12.

7. The hydro-fluorocompound of claim 4, selected from the group consisting of:
CF₃CH₂—O—(CH₂)₂—O—CF₂—CF₂—COOXₐ,
CF₃CH₂—O—(CH₂)₄—O—CF₂—CF₂—COOXₐ,
CF₃CH₂—O—(CH₂)₆—O—CF₂—CF₂—COOXₐ,
CF₃CH₂—O—(CH₂)₈—O—CF₂—CF₂—COOXₐ,
wherein Xₐ is H, a monovalent metal or an ammonium group of formula —N(R′ₙ)₄, wherein each of R′ₙ, equal to or different from each other, independently represents a hydrogen atom or a C₁₋₆ hydrocarbon group.

8. A process for manufacturing a hydro-fluorocompound according to claim 1.

9. A process according to claim 8 for manufacturing the hydro-fluorocompound of formula (IV-A):
R_{f}O—(CH₂)ₚ—O—CH₂—CF(X)—COOXₐ
wherein p=2, said process comprising
reacting an alcohol of formula R_{f}OH, with ethylene carbonate to obtain a hydroxyl derivative of formula R_{f}O—(CH₂)₂—OH, and
reacting the hydroxyl derivative with a fluorinated oxetane derivative of formula: 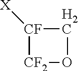
wherein X is F or CF₃, typically with 2,2,3,3-tetrafluorooxetane, to yield, after hydrolysis and neutralization, the carboxylic derivative R_{f}O—(CH₂)₂—O—CH₂—CFX—COOH, which is optionally salified, as required.

10. A process according to claim 8 for manufacturing the hydro-fluorocompound of formula (IV-A):
R_{f}O—(CH₂)ₚ—O—CH₂—CF(X)—COOXₐ
comprising reacting a ω-halo-hydroxy-derivative of formula Hal-(CH₂)ₚ—OH, wherein Hal is a halogen, and p is an integer of 1 to 12, with a fluorinated oxetane derivative of formula: 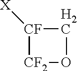
wherein X is F or CF₃, typically with 2,2,3,3-tetrafluorooxetane, to yield a derivative of formula Hal-(CH₂)ₚ—OCH₂—CF(X)—C(O)—O—(CH₂)ₚ-Hal, and subsequently reacting said Hal-(CH₂)ₚ—OCH₂—CF(X)—C(O)—O—(CH₂)ₚ-Hal with the alcoholate form of a fluorinated alcohol of formula R_{f}—OH, to provide, after hydrolysis and neutralization, the carboxylic derivative of formula R_{f}—O—(CH₂)ₚ—OCH₂—CF(X)—COOH which is optionally salified, as required.

11. A process according to claim 8 for manufacturing the hydro-fluorocompound of formula (IV-B):
R_{f}O—(CH₂)ₚ—O—CF₂—CF₂—COOXₐ
said process comprising:
reacting a fluoroalcohol of formula R_{f}OH with a ω-halo-hydroxy-derivative of formula Hal-(CH₂)ₚ—OH, wherein Hal is a halogen, typically Cl, p is an integer of 1 to 12, to yield corresponding adduct of formula R_{f}O—(CH₂)ₚ—OH;
reacting said adduct with a mixture of tetrafluoro ethylene and an alkylcarbonate to yield, after hydrolysis, corresponding carboxylic derivative of formula R_{f}O—(CH₂)ₚ—O—CF₂CF₂—COOH, which is optionally salified, as required can be further salified if needed.

12. A method for making a fluoropolymer comprising aqueous emulsion polymerization of one or more fluorinated monomers in an aqueous medium comprising at least one hydro-fluorocompound according to claim 1.

13. The method of claim 12, wherein the one or more fluorinated monomers comprises vinylidene fluoride (VDF) optionally in combination with one or more fluorinated monomers different from VDF and wherein the method produces a thermoplastic vinylidene fluoride polymer.

14. An aqueous fluoropolymer dispersion comprising a hydro-fluorocompound according to claim 1.

15. The hydro-fluorocompound of claim 1, wherein:
Xₐ is H, a monovalent alkaline metal or an ammonium group of formula —N(R′ₙ)₄, wherein each of R′ₙ, equal to or different from each other, independently represents a hydrogen atom or a C₁₋₆ alkyl group; and
X is F.

16. The hydro-fluorocompound of claim 2, wherein:
R_{f} is a group of formula R′_{f}—CH₂—, wherein R′_{f} is a C₁-C₃ perfluorinated group, optionally comprising one or more ethereal oxygens;
X is F;
Xₐ is H, a monovalent alkaline metal or an ammonium group of formula —N(R′ₙ)₄, wherein each of R′ₙ, equal to or different from each other, independently represents a hydrogen atom or a C₁₋₆ alkyl group; and
p is an integer of 2 to 10.

17. The hydro-fluorocompound of claim 3, wherein:
X is F;
Xₐ is H, a monovalent alkaline metal or an ammonium group of formula —N(R′ₙ)₄, wherein each of R′ₙ, equal to or different from each other, independently represents a hydrogen atom or a C₁₋₆ alkyl group;
p is an integer of 2 to 10, and
R′_{f} is a C₁-C₃ perfluorinated group, optionally comprising one or more ethereal oxygens.

18. The hydro-fluorocompound of claim 4, wherein:
R_{f} is a group of formula R′_{f}—CH₂—, wherein R′_{f} is a C₁-C₃ perfluorinated group, optionally comprising one or more ethereal oxygens;
Xₐ is H, a monovalent alkaline metal or an ammonium group of formula —N(R′ₙ)₄, wherein each of R′ₙ, equal to or different from each other, independently represents a hydrogen atom or a C₁₋₆ alkyl group; and
p is an integer of 2 to 10.

19. The hydro-fluorocompound of claim 6, wherein:
R_{f} is a group of formula R′_{f}—CH₂—, wherein R′_{f} is a C₁-C₃ perfluorinated group, optionally comprising one or more ethereal oxygens;
Xₐ is H, a monovalent alkaline metal or an ammonium group of formula —N(R′ₙ)₄, wherein each of R′ₙ, equal to or different from each other, independently represents a hydrogen atom or a C₁₋₆ alkyl group; and
p is an integer of 2 to 10.

20. The hydro-fluorocompound of claim 7, wherein Xₐ is H.
